(19) United States
(12) Plant Patent Application Publication
(10) Pub. No.: US 2026/0293785 P1
(43) Pub. Date: Sep. 24, 2026

(54) Garden Rose Plant Named 'VLR17004'

(50) ;

(71) Applicant: VINELAND RESEARCH AND INNOVATION CENTRE, Vineland Station (CA)

(72) Inventor: Daryl SOMERS, Vineland Station (CA)

(73) Assignee: VINELAND RESEARCH AND INNOVATION CENTRE, Vineland Station, ON (CA)

(21) Appl. No.: 19/436,028

(22) Filed: Dec. 30, 2025

(30) Foreign Application Priority Data
Mar. 18, 2025 (CA) 25-11981

(51) Int. Cl. A01H 6/74; A01H 5/02

(52) U.S. Cl. CPC A01H 6/749; A01H 5/02
USPC PLT/107

(57) ABSTRACT

A new and distinct variety of garden rose plant, referred to by its cultivar name, ‘VLR17004’, is described. The new plant variety forms in abundance on a substantially continuous basis double petalled pink to cream-colored blossoms. The growth habit is semi-upright, shrub. Semi-glossy, dark green foliage is formed. Additionally, the new plant variety is particularly well suited for growing as distinctive ornamentation in the landscape.

Latin name of genus and species of plant claimed: Rosa hybrida.

Variety denomination: ‘VLR17004’.

### CROSS REFERENCE TO RELATED APPLICATIONS

This application claims priority to Canadian Plant Breeders' Rights Application No. 25-11981, which was filed on Mar. 18, 2025, the contents of which are hereby incorporated by reference for all purposes.

### BACKGROUND OF THE INVENTION

The new plant variety of garden rose plant of the present invention was created by controlled breeding in 2017 in Vineland Station, Ontario, Canada by artificial pollination wherein two parents were crossed which previously had been studied in the hope that they would contribute the desired characteristics. The female parent (i.e., the seed parent) of the new plant variety was ‘V90’ (not patented). The male parent (i.e., the pollen parent) of the new plant variety was ‘V109’ (not patented).

The parentage of the new plant variety can be summarized as follows:
‘V90’ x ‘V109’

The seeds resulting from the above pollination were sown and small plants were obtained which were physically and biologically different from each other. Selective study resulted in the identification of a single plant of the new variety.

The new variety has been found to undergo asexual propagation at Vineland Station, Ontario, Canada by a number of routes such as vegetative cuttings. Asexual propagation techniques in Vineland Station, Ontario, Canada, such as vegetative cuttings, have shown that the characteristics of the new variety are homogeneous, stable, and strictly transmissible by such asexual propagation from one generation to another. Accordingly, the new variety undergoes asexual propagation in a true-to-type manner.

### SUMMARY OF THE INVENTION

It was found that the new variety of garden rose plant of the present invention possesses the following combination of characteristics:
(a) abundantly and substantially continuously forming double petalled pink to cream-colored blossoms,
(b) exhibiting a semi-upright, shrub growth habit, and
(c) forming semi-glossy, dark green foliage.

The new plant variety well meets the needs of the horticultural industry. It can be grown to advantage as distinctive ornamentation in parks, gardens, public areas, and residential landscapes. Accordingly, it is particularly well suited for growing in the landscape.

The new plant variety can be readily distinguished from its ancestors. Moreover, the new plant variety can be readily distinguished from non-parental related similar varieties. For example, the new variety displays bi-color pink to cream-colored flowers and forms dark green, semi-glossy foliage, whereas the ‘FRYcentury’ variety (U.S. Plant Pat. No. 15,334; DAY BREAKER™) displays larger bi-color pink to yellow-colored flowers, grows to a slightly taller height, forms smaller leaves and larger leaflets, and forms glossier foliage compared to the new plant variety. Additionally, the new plant variety has improved winter hardiness up to USDA Zone 4b compared to the ‘FRYcentury’ variety which is cold hardy up to USDA Zone 6b.

The new plant variety has been named the ‘VLR17004’ variety.

### BRIEF DESCRIPTION OF THE PHOTOGRAPHS

The accompanying photographs show, as nearly true as it is reasonably possible to make the same in a color illustration of this character, typical specimens of the new plant variety and blossoms of the new plant variety. The illustrated rose plant of the new variety was grown in Vineland Station, Ontario, Canada.

FIG. 1 illustrates a side-by-side view of the flower buds of ‘VLR17004’, left, and ‘FRYcentury’, right.

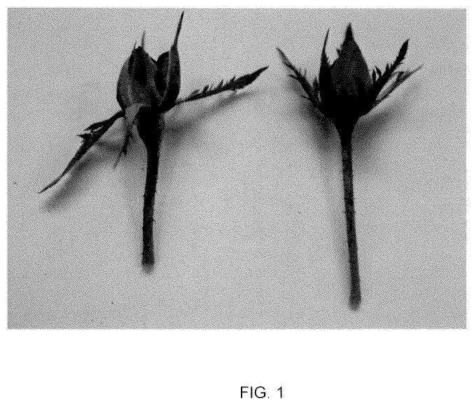

FIG. 2 illustrates a side-by-side top view of opened flowers of ‘VLR17004’, left, and ‘FRYcentury’, right.

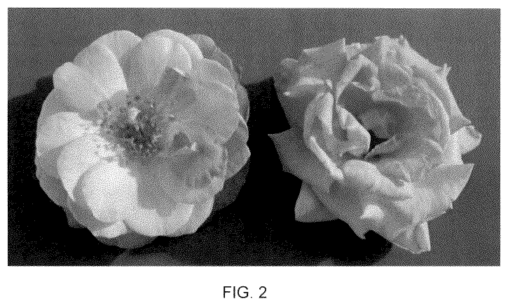

FIG. 3 illustrates a side-by-side view of the specimens of the ‘VLR17004’, left, and ‘FRYcentury’, right, plants growing in containers displaying flowers at varying points of opening.

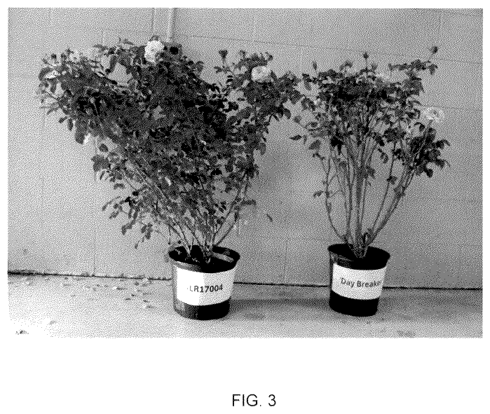

### DETAILED BOTANICAL DESCRIPTION

The chart used in the identification of colors is that of The Royal Horticultural Society (The R.H.S. Colour Chart, 2007 edition). The terminology which precedes reference to the chart has been added to indicate the corresponding color in more common terms. The R.H.S. Colour Chart designation used herein represents the closest color observed on the majority of the specified botanical features. The description is based on a two-year-old specimen of the new variety, observed during September 2025, while growing in a three-gallon container in Vineland Station, Ontario, Canada.
Botanical classification: Rosa hybrida cultivar ‘VLR17004’.
Commercial classification: Garden Rose.
Plant:
   Habit.—Semi-upright, shrub.
   Height.—Approximately 70.6 cm on average during second flush.
   Width.—Approximately 70 cm on average.
Branches:
   Stem color.—Old wood: Green Group 138B. — young stems: Green Group 138B.
   Length.—Main stems: approximately 70 cm on average. — secondary stems: approximately 54 cm on average.
   Diameter.—Main stems: approximately 1.2 cm on average. — secondary stems: approximately 8 mm on average.
   Internode length.—Main stems: approximately 3.5 cm on average. — secondary stems: approximately 3.5 cm on average.
   Texture.—Main stems: woody with prickles. — secondary stems: woody with few prickles.
   Anthocyanin coloration of young shoots.—Strong, Red Group 53B.
   Prickles.—Amount: medium, typically 6 per internode. — young prickles: length is approximately 5 mm on average; width is approximately 3 mm on average at point of attachment; and color is reddish, Orange-Red Group 34D. — old prickles: length is approximately 1.1 cm on average; width is approximately 8 mm on average at point of attachment; and color is Orange Group 24C.
   Flowering shoots.—Number of flowering laterals: medium, typically 3. — number of flowers (only shoots with no flowering laterals): very few, typically 1. — number of flowers (only shoots with flowering laterals): very few, typically 2.
Leaflets:
   Number.—Mostly five.
   Shape of terminal leaflet.—Medium elliptic; apex is acuminate; and base is obtuse.
   Shape of lateral leaflet.—Elliptical; apex is elliptical; and base is obtuse.
   Arrangement.—Odd-pinnate.
   Margin.—Serrated.
   Texture.—Upper surface: smooth. — under surface: bit rough.
   Venation pattern.—Reticulate.
   Undulation.—Medium.
   Glossiness of upper side.—Medium.
   Glossiness of lower side.—Dull.
   Intensity of green color on upper side.—Dark.
   Intensity of green color on lower side.—Light green.
   Anthocyanin coloration of newly opened leaflets.—Medium, Red Group 47D.
   Size.—Terminal leaflet: Length is approximately 5.1 cm on average; width is approximately 3.1 cm on average. — lateral leaflets: length is approximately 4.1 cm on average; width is approximately 3.0 cm on average. — Leaf: length is approximately 12.1 cm on average; width is approximately 8.9 cm on average.
   Petiole.—Texture: few thorns. — length: approximately 2.2 cm on average. — diameter: approximately 1.0 mm on average. — color: Green Group N138B.
   Rachis.—Texture: few thorns. — length: approximately 4.2 cm on average. — diameter: approximately 0.6 mm on average. — color: Green Group 143C.
   Petiolules.—Texture: smooth. — length: approximately 1.0 mm on average. — diameter: approximately 0.8 mm on average. — color: Green Group 138C.
Foliage:
   Young foliage.—Upper surface color: Red Group 53B with Green Group 137C venation. — under surface color: Red Group 53D with Green Group 138B venation.
   Old foliage.—Upper surface color: Green Group N137C with Green Group 138A venation. — under surface color: Green Group 137C with Green Group 139C venation.
   Anthocyanin coloration of mature leaves.—Absent.
Inflorescence:
   Number of flowers.—About 14 blooms on average on a plant at once.
   Number of blossoms per stem or in a cluster.—Typically 1 blooms per stem on average.
   Peduncle.—Color: Green Group 138B. — diameter: approximately 2.5 mm on average. — length: approximately 4.9 cm on average. — surface texture: fine soft prickles.
   Sepals.—Upper surface color and texture: lighter green with fine hairs, Green Group 138D. — under surface color and texture: smooth, Green Group 138B. — size: length is approximately 2.2 cm on average; width is approximately 7 mm on average. — margin: smooth. — undulation: absent. — shape: pointed. — apex: pointed. — extensions: weak.
   Buds.—Shape: medium ovate. — color (just before the separation of the sepals): Red Group 43D.
   Flower.—Form: double. — shape: round. — profile: upper part is flattened convex; lower part is flattened convex. — diameter: approximately 7.7 cm on average. — depth: approximately 3.4 cm on average — duration: on the plant approximately 7 days. — color group: multicolored. — color of the center when viewed from above: pink, Orange-Red Group N66C. — petals reflex one by one: yes.
   Fragrance.—Absent or weak.
   Petal.—Number: approximately 39 on average. — length: approximately 4.0 cm on average. — width: approximately 3.4 cm on average. — overall shape: rounded. — margin: wavy. — reflexing of margin: absent or very weak. — undulation: absent or very weak. — apex shape: rounded. — base shape: narrow down. — texture (both surfaces): smooth. — incisions: absent or very weak. — density of petals: medium. — number of colors on upper side (basal spot excluded): two. — intensity of color: lighter towards the base. — main color on the upper side: Red Group 38B. — basal spot on upper side: small, with a color of light yellow, Yellow Group 9B. — secondary color: Yellow Group 12C distributed at base. — main color on the lower side: Red Group 39C.
   Stamen.—Number: 68. — anthers: color is Orange Group 24A; length is approximately 1.0 mm on average; width is approximately 0.6 mm on average; and shape is ellipsoid. — filaments: length is approximately 6 mm on average, diameter is approximately 0.4 mm on average, and color of filament of outer stamen is light yellow, Yellow Group 13C.
   Pistils.—Number: 48. — style: length is approximately 2 mm on average, diameter is approximately 0.3 mm on average, and color is Yellow Group 3C. — stigma: length is approximately 0.5 mm on average, width is approximately 0.5 mm on average; shape is capitate; color is Yellow Group 4A.
   Receptacle.—Diameter: approximately 7 mm on average. — depth: approximately 7.5 mm on average. — color: Green Group 143A.
   Pollen.—Amount: abundant. — color: Yellow-Orange Group 21C.
   Seed vessel.—Size at petal fall: medium, length is approximately 9 mm on average, diameter is approximately 9 mm on average.
   Hip.—Shape in longitudinal section: pear-shaped. — color at mature stage: orange.
Development:
   Vegetation.—Semi-glossy, dark green.
   Blossoming.—Typically May to October.
   Resistance to diseases.—Resistant to black spot (Diplocarpon rosae).
   Hardiness.—Hardy to USDA Zone 4b.

The new ‘VLR17004’ variety has not been observed under all possible environmental conditions to date. Accordingly, it is possible that the phenotypic expression may vary somewhat with changes in light intensity and duration, cultural practices, and other environmental conditions.

I claim:

1. A new and distinct variety of garden rose plant named ‘VLR17004’ characterized by the following combination of characteristics:
(a) abundantly and substantially continuously forming double petalled pink to cream-colored blossoms,
(b) exhibiting a semi-upright, shrub growth habit, and
(c) forming semi-glossy, dark green foliage;
substantially as herein shown and described.
